(19) United States
(12) Patent Application Publication
(10) Pub. No.: US 2012/0209339 A1
(43) Pub. Date: Aug. 16, 2012

(54) DEVICE FOR SPINAL FUSION

(76) Inventors: Daniel Scodary, Bridgeton, MO (US); Paul M. Sand, Redwood City, CA (US)

(21) Appl. No.: 13/025,988

(22) Filed: Feb. 11, 2011

(51) Int. Cl. A61B 17/56

(52) U.S. Cl. 606/86 R

(57) ABSTRACT

An improved device for filling a void created in the lamina during spinal surgery including a top layer, a bottom layer and a body portion. The body portion is attached to the top layer and the bottom layer and includes a compliant mesh portion. The compliant mesh portion includes a bone material with bone morphogenetic protein (BMP) growth factor entrained thereon. The body portion is adapted to be conformable to a void left in a vertebral space after a surgeon has performed a laminectomy or hemi-laminectomy procedure. The flexible top layer has a periphery larger than that of the bottom layer such that the body portion has a section that is oblique or perpendicular to another section of the body portion or the top layer to define a flange. A method of using the device is also disclosed.

### FIELD OF THE INVENTION

The present invention relates generally to intervertebral defect devices, and more particularly, to an intervertebral defect device for insertion into an intervertebral space using minimally invasive techniques.

### BACKGROUND OF THE INVENTION

Referring to prior art FIGS. 1 and 2, the spine 120 is a flexible column of vertebrae 100 held together by muscles, ligaments and tendons. The spine 120 extends from the cranium (not shown) to the coccyx 126, encasing a spinal cord 128 and forming the supporting axis of the body. The spinal cord 128 is a thick bundle of nerve tissue that branch off to various areas of the body for the purposes of motor control, sensation, and the like. The spine 120 includes seven cervical vertebrae, twelve thoracic vertebrae, five lumbar vertebrae, L^{I}-L^{V}, five sacral vertebrae, S^{I}-S^{V}, and three coccyx vertebrae 126. The sacral and coccyx vertebrae are each fused, thereby functioning as a single unit. FIG. 2 shows the lumbar region 122, the sacral region 124 and the coccyx 126 of the spine 120 and that the vertebrae 100 are stacked one upon another. The top portion 100a and bottom portion 100b of each vertebrae 100 is slightly concave. The opposing concave vertebral surfaces form the intervertebral space 121 in which an intervertebral disk (not shown) resides. Each of the intervertebral disks has a soft core referred to as a nucleus pulposus or nucleus (not shown).

In FIG. 1, directional arrow 101a is pointing in the posterior direction and directional arrow 101b is pointing in the anterior direction. FIG. 1 shows that each vertebrae 100 includes a body 106 in the innermost portion, a spinal canal 108 and a spinous process 102 at the posterior-most end of the vertebra 100. The vertebrae 100 are substantially similar in composition, but vary in size from the larger lumbar vertebrae to the smallest coccyx vertebrae 126. Each vertebrae 100 further includes two transverse processes 104 located on either side and a protective plate-like structure referred to as a lamina 110. Nerves from the spinal cord 128 pass through the spinal canal 108 and foramina 111 to reach their respective destinations within the body.

The natural aging process can cause a deterioration of the intervertebral disks, and therefore, their intrinsic support strength and stability is diminished. Sudden movements may cause a disk to rupture or herniate. A herniation of the disk is primarily a problem when the nucleus pulposus protrudes or ruptures into the spinal canal 108 placing pressure on nerves which in turn causes spasms, tingling, numbness, and/or pain in one or more parts of the body, depending on the nerves involved. Further deterioration of the disk can cause the damaged disk to lose height and as bone spurs develop on the vertebrae 100, result in a narrowing of the spinal canal 108 and foramen 111, and thereby causes pressure on the nerves emanating from the spinal cord 128.

Presently, there are several techniques, in addition to non-surgical treatments, for relieving the symptoms related to intervertebral disk deterioration. Surgical options include chemonucleolysis, laminectomy, diskectomy, microdiskectomy, and spinal fusion.

A laminectomy, or hemi-laminectomy, is performed to decompress the spinal canal by open surgical techniques under general anesthesia. In this procedure, the lamina 110, (the bone that curves around and covers the spinal canal 108 as shown in FIG. 1), and any disk tissue causing pressure on a nerve or the spinal canal 108, are partially removed. This technique is invasive and traumatic to the body, and therefore requires an extended recovery time of about five weeks and a hospital stay of a few days. In addition to the trauma inflicted on the body from even a successful surgery, previously there were increased risks of future problems due to the removed portion of the lamina 110 which is no longer in place to support and protect the spinal canal 108 at the area where the surgery took place. Further, the vertebrae 100 are at risk of shifting due to the lack of support in the structure.

What is needed, but not provided in the prior art, is a stand alone vertebral defect device that can be inserted into the void created during a hemi-laminectomy to provide stability of the spine, promote bone growth and protect the protect the patient's nerves during and after recovery.

### BRIEF SUMMARY OF THE INVENTION

An improved device for filling a void created in the lamina during spinal surgery including a top layer, a bottom layer and a body portion. The body portion is attached to the top layer and the bottom layer and includes a compliant mesh portion. The compliant mesh portion includes a bone material with bone morphogenetic protein (BMP) growth factor entrained thereon. The body portion is adapted to be conformable to a void left in a vertebral space after a surgeon has performed a laminectomy or hemi-laminectomy procedure. The flexible top layer has a periphery larger than that of the bottom layer such that the body portion has a section that is oblique or perpendicular to another section of the body portion or the top layer to define a flange. A method of using the device is also disclosed.

### BRIEF DESCRIPTION OF THE SEVERAL VIEWS OF THE DRAWINGS

FIG. 1 is a top sectional view of a human vertebra;

FIG. 2 is a side sectional view of a portion of a human spine;

FIG. 3 is a side view of a device according to a preferred embodiment of the present invention;

FIG. 4 is an end view of a device according to a preferred embodiment of the present invention;

FIG. 5 is a bottom view of a device according to a preferred embodiment of the present invention;

FIG. 6 is a top view of a device according to a preferred embodiment of the present invention;

FIG. 7 is a bottom perspective view of a device according to a preferred embodiment of the present invention;

FIG. 8 is a top perspective view of a device according to a preferred embodiment of the present invention;

FIG. 9 is a posterior view of a spinal column having a void resulting from a hemi-laminectomy therein;

FIG. 10 is a posterior view of a spinal column having a void resulting from a hemi-laminectomy filled by a device according to an embodiment of the present invention; and

FIG. 11 is an axial view of a spinal column having a void resulting from a hemi-laminectomy filled by a device according to an embodiment of the present invention.

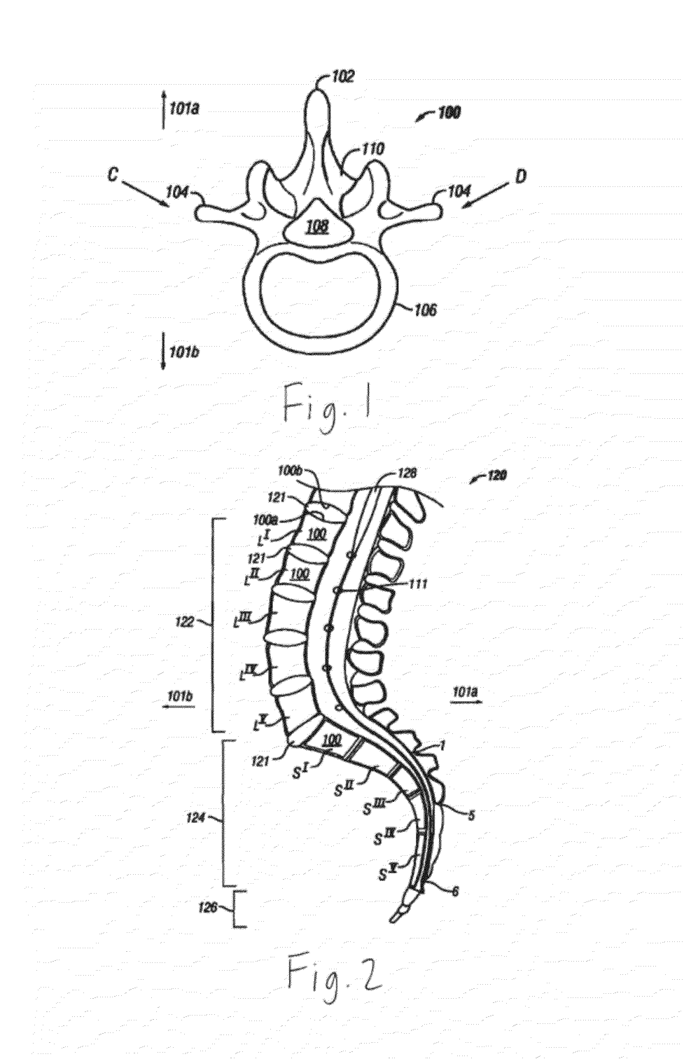

### DESCRIPTION OF THE PREFERRED EMBODIMENT

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The present invention comprises an improved device for filling a void created by a laminectomy or hemi-laminectomy surgical procedure. The device comprises a flexible top and bottom layer and a gel-suspended bone portion between the top and bottom layer to conform to the laminectomy void.

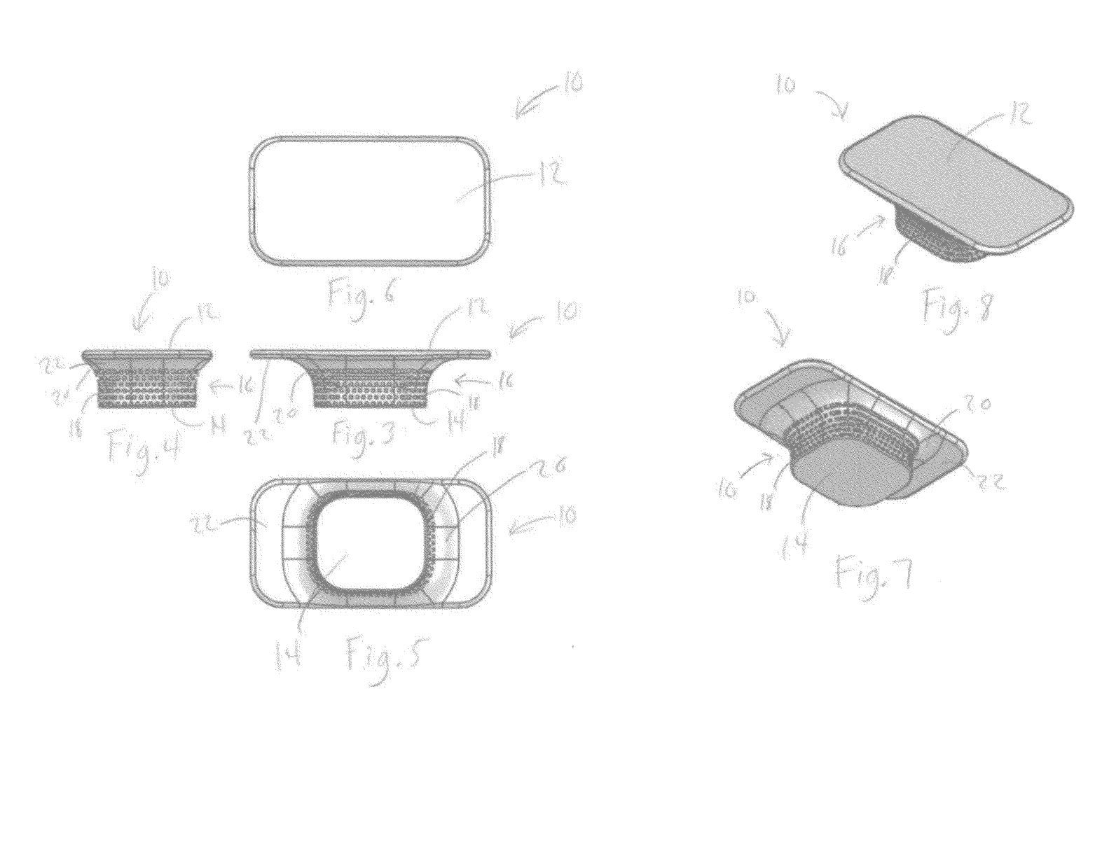

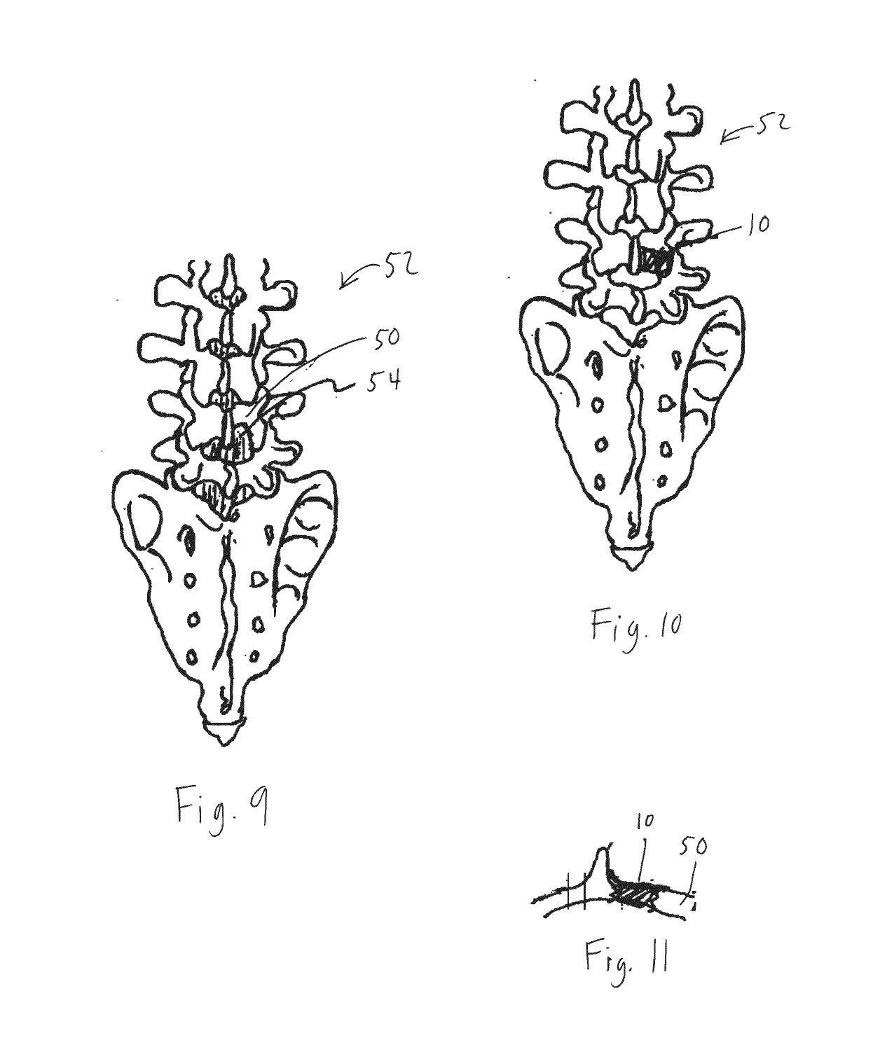

In that regard and referring to FIGS. 3 through 8, there is shown a device 10 according to the present invention. The device 10 comprises a top layer 12 and a bottom layer 14 comprising a woven or knitted mesh or netting material. The material is preferably bio-absorbable or partially absorbable, although non-absorbable materials may be used consistent with the present invention. Examples of absorbable materials that may be used are: polylactic acid, polyglycolic acid, polydioxanone, poliglecaprone-25, catgut and silk. Examples of non-absorbable materials that may be used are: polypropylene and Dacron/polyester. Partially absorbable materials may be selected from combinations of absorbable and non-absorbable materials.

The top layer 12 and bottom layer 14 are flexible to conform to the anatomy of the patient. The top layer further has an anti-adhesion coating to prevent the posterior tissue and muscle from adhere to the top layer 12. Likewise, the bottom layer 14 further includes a coating with anti-adhesion properties to prevent the spinal cord and spinal tissue from adhering to the bottom layer 14.

Attaching the top layer 12 and bottom layer 14 is a body portion 16. The body portion 16 comprises a first, void filler section 18 attached to a second, transition section 20 further attached to a third, flange section 22.

The first section 18 has an outer circumference that general corresponds to the outer circumference of the bottom layer 14 and adheres the bottom layer 14 to the body portion 16. It is preferably a compliant mesh portion having bone material with bone morphogenetic protein (BMP) growth factor and generally conforms to the void left in a vertebrae after a surgeon has performed a laminectomy or hemi-laminectomy procedure. As mentioned, the compliant mesh will deform to conform to the void and contact the remaining bone from the vertebrae to promote bone regrowth in the void. In that regard, the mesh having BMPs create an osteinductive environment and the mesh construction create an osteoconductive mold upon which new bone may grow.

The third section 22 is generally perpendicular to the first section 18 and is compliant to the void over which it is placed. The third section 22 expands outwardly from the first section 18 and forms a flange having a coating that adheres to bone. The third section 22 contacts the remaining bone surrounding void made in the vertebra and with the bone-adhering coating adheres the entire device 10 to the vertebra and within the void. The flange of the third section 22 is also compliant to match the shape of the patient's vertebra to provide better adherence thereto. The third section 22 further attaches the top layer 12 to the device 10.

The second section 20 is preferably arcuate and attached the first section 18 to the third section 22. The second section 20 may be either mesh, as in the first section 18, or non-mesh as in the third section 22, but most preferably transitions from a mesh to a non-mesh construction.

The device 10 of the present invention is utilized by a surgeon who has removed a portion 54 of a vertebra 50 of a patient's spine 52, as shown in FIG. 9, to relieve pressure upon a patient's nerves caused by, for example, a degenerative condition. Referring to FIGS. 10 and 11, the device 10 of the present invention is inserted into the void and adhered to the void to regrow bone within the void thereby returning stability to the spine and protecting the spinal canal. As can be seen in FIG. 11, the flange of the third section 22 deforms to comply with the shape of the patient's vertebra, and in this case curves to adhere to the spinous process.

Exemplary embodiments of the present invention are described above in detail. The device is not limited to the specific embodiment described herein. While the invention has been described in terms of a specific embodiment, those skilled in the art will recognize that the invention can be practical with modification within the spirit and scope of the claims. In view of the above, it will be seen that the advantages of the present invention have been achieved and other advantageous results have been obtained.

We claim:

1. An improved device for filling a void created in the lamina during spinal surgery comprising:
a top layer;
a bottom layer
a body portion attached to the top layer and the bottom layer, the body portion comprising a compliant mesh portion comprising bone material with bone morphogenetic protein (BMP) growth factor entrained thereon, the body portion adapted to be conformable to a void left in a vertebrae after a surgeon has performed a laminectomy or hemi-laminectomy procedure; and
the flexible top layer having a periphery larger than that of the bottom layer such that the body portion has a section that is oblique or perpendicular to another section of the body portion or the top layer to define a flange.

2. The device of claim 1 wherein the top layer is a generally flexible mesh material.

3. The device of claim 1 wherein the bottom layer is a generally flexible mesh material.

4. The device of claim 1 wherein the body portion comprises a first, void filler section attached to a second, transition section further attached to a third, flange section.

5. The device of claim 1 wherein at least one of the top and bottom portions comprise an at least partially bio-absorbable material.

6. The device of claim 5 wherein the at least partially bio-absorbable material is selected from the group consisting of: polylactic acid, polyglycolic acid, polydioxanone, poliglecaprone-25, catgut and silk.

7. The device of claim 1 wherein at least one of the top and bottom portions comprise a non bio-absorbable material.

8. The device of claim 7 wherein the at least partially non bio-absorbable material is selected from the group consisting of: polypropylene and Dacron/polyester

9. The device of claim 1 wherein at least one of the top layer and bottom layer comprises an anti-adhesion coating

10. The device of claim 1 wherein the flange is generally flexible and comprises a coating that adheres to bone.

11. An improved device for filling a void created in the lamina during spinal surgery comprising:
a generally flexible top layer;
a generally flexible bottom layer
a body portion attached to the top layer and the bottom layer, the body portion comprising a compliant mesh portion having bone material, the body portion adapted to be conformable to a void left in a vertebrae after a surgeon has performed a laminectomy or hemi-laminectomy procedure; and
the flexible top layer having a periphery larger than that of the bottom layer such that the body portion has a section that is oblique or perpendicular to another section of the body portion and the top layer to define a flange.

12. The device of claim 11 wherein the body portion comprises a first, void filler section attached to a second, transition section further attached to a third, flange section.

13. The device of claim 11 wherein at least one of the top and bottom portions comprise an at least partially bio-absorbable material.

14. The device of claim 13 wherein the at least partially bio-absorbable material is selected from the group consisting of: polylactic acid, polyglycolic acid, polydioxanone, poliglecaprone-25, catgut and silk.

15. The device of claim 11 wherein at least one of the top and bottom portions comprise a non bio-absorbable material.

16. The device of claim 15 wherein the at least partially non bio-absorbable material is selected from the group consisting of: polypropylene and Dacron/polyester

17. The device of claim 11 wherein at least one of the top layer and bottom layer comprises an anti-adhesion coating

18. The device of claim 11 wherein the flange is generally flexible and comprises a coating that adheres to bone.

19. A method of performing a laminectomy or hemilaminectomy surgical procedure comprising the steps of:
removing a portion of the patient's spinal lamina to create a void;
inserting a device into the void, the device comprising:
a generally flexible top layer;
a generally flexible bottom layer
a body portion attached to the top layer and the bottom layer, the body portion comprising bone material, the body portion conformable to the void; and
retaining the device within the void with a bone-adhering adhesive.

20. The method of claim 19 wherein the body portion comprises a first, void filler section attached to a second, transition section further attached to a third, flange section.

21. The method of claim 19 wherein the top and bottom layers comprise an at least partially bio-absorbable material selected from the group consisting of: polylactic acid, polyglycolic acid, polydioxanone, poliglecaprone-25, catgut and silk.

22. The method of claim 19 wherein the top and bottom layers comprise a non bio-absorbable material selected from the group consisting of: polypropylene and Dacron/polyester

23. The method of claim 19 wherein at least one of the top layer and bottom layer comprises an anti-adhesion coating

24. The method of claim 19 wherein the flange is generally flexible and comprises a coating that adheres to bone.
